(19) United States
(12) Patent Application Publication
(10) Pub. No.: US 2023/0295551 A1
(43) Pub. Date: Sep. 21, 2023

(54) Microbe-Aided Perfusion System for Metallic Nanoparticle Production

(71) Applicant: Northeastern University, Boston, MA (US)

(72) Inventors: Linh TRUONG, Boston, MA (US); David Medina CRUZ, Boston, MA (US); Thomas WEBSTER, Barrington, RI (US)

(21) Appl. No.: 18/021,447

(22) PCT Filed: Aug. 17, 2021
(86) PCT No.: PCT/US2021/046387

§ 371 Date: Feb. 15, 2023

Related U.S. Application Data
(60) Provisional application No. 63/066,795, filed on Aug. 17, 2020.

(51) Int. Cl. C12M 1/00; C12M 1/12; C12M 1/04; C12N 1/20

(52) U.S. Cl. CPC C12M 29/10; C12M 23/04; C12M 23/34; C12M 25/02; C12M 23/24; C12N 1/20

(57) ABSTRACT

A continuous perfusion bioreactor for microbial synthesis of metallic nanoparticles provides synthesis of nanoparticles without washout of the microbial cells. The perfusion bioreactor includes an inlet fluidically coupled to a holding compartment with a first flow barrier, which disperses the medium into a reaction compartment suitable for culture of microbial cells which synthesize nanoparticles. A second flow barrier in the reaction compartment prevents washout of the microbial cells but enables collection of the nanoparticles suspended in depleted nutrient medium.

### CROSS-REFERENCE TO RELATED APPLICATIONS

This application claims priority to U.S. Provisional Application No. 63/066,795, filed 17 Aug. 2020, which is incorporated by reference herein in its entirety.

### BACKGROUND

Metallic nanoparticles (NPs) represent a new frontier for many industries, especially healthcare. Their antioxidant (L. Valgimigli, et al., 2018), antimicrobial (H. Zazo, et al., 2017), and anticancer (Mei & Wu, 2017) properties have been shown to have therapeutic applications. With demand for NPs on the rise, it is important to find methods to produce NPs in an eco-friendly, cost-effective, efficient, and consistent manner. Metallic NPs can be produced using bacterial cells (A. Fariq, et al., 2017), lowering the industrial and energy waste compared to conventional chemical methods. However, the current processes for bacterial synthesis of metallic nanoparticles require extensive human labor, resulting in unreliable results between batches, variance in yield, and variance in purity, with significant risk of contamination (M. L. Verma, et al., 2019). Therefore, there is a need for new methods and devices for improved production of metallic NPs and products using microbial cells.

### SUMMARY

The present technology provides devices and methods for synthesis of nanoparticles (NPs) using microbial cells. The technology can be practiced using any cells capable of forming nanoparticles by the chemical conversion of one or more metal salt into metallic nanoparticles, microbial cells such as bacterial cells are preferred. Preferably, the chemistry performed is the reduction of metal salts form solid metal nanoparticles or microparticles. The technology provides a specialized perfusion bioreactor with separate and specialized components for the culture of microbes (cells), the synthesis of NPs by those microbes, and the subsequent recovery of the NPs produced using a continuous flow process. The technology can provide a continuous, automated synthesis of NPs, without requiring constant or intermittent replenishment of the microbial cells during a biosynthesis of the NPs. The devices and methods can prevent the problematic co-elution of microbial cells from a culture along with the NPs, a problem often referred to as cell washout. The methods optionally utilize adhesion or a semi-adhesion of the cells to the main perfusion reactor, which can ensure efficient separation of the NPs from the microbial cells. The synthesized NPs and reacted medium flows to a collection port after passing a flow barrier that retains the microbial cells in the main perfusion reactor, while allowing the NPs to pass on to collection cell-free. A filter within the collection chamber or at the collection port can further separate the NPs from the microbial cells.

The present methods and devices can be used to synthesize metallic nanoparticles containing or consisting of any metal element, including for example Ag, Pt, Pd, Se, and any combination thereof, or their alloys with other metals, and may also include non-metal elements. Metallic nanoparticles and the reaction conditions and chemistry for their synthesis by microbial cells can be found, for example, in WO 2018/218091 A1 and WO 2020/206459 A1, which are hereby incorporated by reference.

The present technology can be further summarized by the following list of features.
1. A perfusion bioreactor for continuous microbial synthesis of metallic nanoparticles, the bioreactor comprising:
   (i) an inlet stage comprising a liquid nutrient medium reservoir fluidically coupled to a pump, the pump fluidically coupled to a perfusion compartment;
   (ii) the perfusion compartment, comprising
      a holding compartment configured for receiving the nutrient medium from the inlet stage, the holding compartment configured for uniform distribution and transfer of the nutrient medium to a reaction compartment;
      the reaction compartment configured for formation of said nanoparticles by microbial cells, the reaction compartment comprising a reaction surface suitable for supporting microbial cells used to synthesize the nanoparticles;
      a collection compartment configured to collect nutrient medium containing the synthesized nanoparticles from the reaction compartment and transfer the medium and nanoparticles to a collection port;
      a first flow barrier disposed between the holding compartment and the reaction compartment, wherein the first flow barrier is operative to uniformly disperse the nutrient medium prior to entry of the medium into the reaction compartment; and
      a second flow barrier disposed between the reaction compartment and the collection compartment, wherein the second flow barrier is operative to retain the microbial cells in the reaction compartment and allow the nanoparticles to flow into the collection compartment.
   (iii) an outlet stage fluidically coupled to the collection port, the outlet stage operative to collect the nanoparticles and depleted nutrient medium from the collection port;
2. The bioreactor of feature 1, wherein the second flow barrier provides a suspension of nanoparticles to the collection compartment that is substantially free of the microbial cells.
3. The bioreactor of feature 2, wherein the second flow barrier provides a suspension of nanoparticles to the collection compartment that comprises less than 1% (weight/weight) microbial cells.
4. The bioreactor of any of the preceding features, wherein the collection compartment comprises one or more filters.
5. The bioreactor of feature 4, wherein the filter comprises pores having an average diameter of about 0.2 μm.
6. The bioreactor of any of the preceding features, wherein the perfusion bioreactor comprises a gas-permeable roof
7. The bioreactor of any of the preceding features, wherein the reaction surface is removable.
8. The bioreactor of any of the preceding features, wherein the collection compartment has a triangular shape converging at an apex to the collection port.
9. The bioreactor of any of the preceding features, wherein the first flow barrier provides a flow of the nutrient medium that is slower than the flow of the liquid medium from the inlet stage.
10. A method for synthesizing metallic nanoparticles, the method comprising the steps of:
   (a) providing the perfusion bioreactor of any of the preceding features, a microbial cell culture, a liquid nutrient medium for growth of the microbial cells, and a metal salt;
   (b) cultivating microbial cells in the reaction compartment of the perfusion bioreactor in the presence of the metal salt under flow of the liquid nutrient medium through the reaction compartment, whereby said metallic nanoparticles are formed in the liquid nutrient medium and collected in the collection chamber; and
   (c) collecting the metallic nanoparticles at the collection port of the collection chamber.
11. The method of feature 10, wherein the microbial cells are bacterial cells.
12. The method of feature 10 or 11, wherein step (b) is performed continuously for at least about one week.
13. The method of any of features 10-12, wherein the nanoparticles collected in step (c) are in form of a suspension of nanoparticles that is essentially free of microbial cells from the reaction compartment.
14. The method of feature 13, wherein the suspension of nanoparticles comprises less than 1% (weight/weight) of said microbial cells.
15. The method of any of features 10-14, further comprising filtering the suspension of nanoparticles.
16. The method of feature 15, wherein the filtering comprises a use of a filter comprising pores with an average diameter of about 0.2 μm.
17. A kit for the synthesis of nanoparticles by microbial cells, the kit comprising the perfusion compartment of the perfusion bioreactor of any of features 1-9, and instructions for performing the method of any of features 10-16.
18. The kit of feature 17, further comprising one or more replacement reaction surfaces of the perfusion compartment.
19. The kit of feature 17 or 18, further comprising the inlet stage and/or outlet stage of the perfusion bioreactor of feature 1.
20. The kit of any of features 17-19, further comprising one or more microbial cell cultures and/or one or more reagents for the production of metallic nanoparticles.

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expression “consisting of” or “consisting essentially of”.

As used herein, “nanoparticle” refers to a particle of any shape with one or more dimensions, including their mean diameter, in the range from about 1 nm to about 999 nm, such as in the range from about 1 nm to about 100 nm.

### BRIEF DESCRIPTION OF THE DRAWINGS

FIG. 1 shows an example of an overall process flow diagram including (1) medium reservoir, containing media, pump 2, tubing 3, lines, or flow channels, main perfusion reactors 4, each with reaction compartment 19 inside, filters 5, collection reservoir 6, and further processing 7 such as post-production processing or transfer to secondary processes. The perfusion system 40 includes the inlet stage 38 (e.g., medium reservoir, pump, and inlet tubing), the main perfusion reactor 4, and the outlet stage 39 (e.g., filter and collection reservoir).

FIG. 2 shows a schematic illustration of a peristaltic pump (prior art).

FIG. 3 shows a side view of an example perfusion reactor 4 including a perfusion compartment 19, which includes holding compartment 8, first flow barrier 9, reactiion compartment 10, second flow barrier 11, and collection compartment 12 with collection port 14. The dimensions shown are represented in inches.

FIG. 4 shows a top view of an embodiment of perfusion compartment 19, including a reaction surface 13, collection compartment 12, and collection port 14. The example are represented in inches.

FIG. 5 shows a right side view of an embodiment of a perfusion reactor 4, including an internal perfusion compartment 19 and including inlet port 15 for introduction of medium. The dimensions are depicted in inches.

FIG. 6 shows a bottom view of an embodiment of perfusion compartment 19, centered on the collection compartment 12 and showing the shape 57 of the portion thereof surrounding the collection port 14. The dimensions are depicted in inches.

FIG. 7 shows an example of a removable reaction surface 17 of a perfusion compartment 19.

FIG. 8 shows an example of roof 18 of perfusion compartment 19.

FIG. 9 shows a 3-dimensional perspective view of frame 16 and removable reaction surface 17 of perfusion compartment 19, without the roof of the compartment removed.

FIG. 10 shows the assembled frame 16 and roof 18 of perfusion compartment 19.

FIG. 11 shows an example of main perfusion reactor 4 including the perfusion compartment's final appearance as viewed from an outside perspective, which includes the frame 16 and roof 18. The inlet tubing 34 and outlet tubing 35 (or collection tubing) are also depicted.

FIG. 12 shows fluid simulation results with 7E-05 m/s inlet velocity of a surface A parallel to the reaction surface, and a velocity magnitude scale is shown at left. The FIG. shows velocity magnitude at the holding compartment 20, velocity magnitude at the reaction compartment 21, velocity magnitude at the collection compartment 22, velocity magnitude at the inlet port 23, and velocity magnitude at the collection port 24, in isometric view.

FIG. 13 shows fluid simulation results with 7E-05 m/s inlet velocity of a surface B parallel to the inlet port and perpendicular to the reaction surface and surface A in right view projection. A velocity magnitude scale is shown at left.

FIG. 14 shows fluid simulation results with 7E-05 m/s inlet velocity of a surface C perpendicular to the reaction surface and the inlet port. The figure shows velocity magnitude at the holding compartment 70, velocity magnitude at the reaction compartment 71, velocity magnitude at the collection compartment 72, velocity magnitude at the inlet port 73, velocity magnitude at the collection port 74, velocity magnitude 75 at the downhill incline 52 of first flow barrier 9, velocity magnitude 76 at the reaction surface, and velocity magnitude 77 at uphill incline 77 of the second flow barrier 11. A velocity magnitude scale is depicted at left.

FIG. 15 shows a wall shear stress simulation using Ansys Fluent software with 7E-05 m/s inlet velocity at the surface parallel to the reaction surface. The simulation includes wall shear stress 25 at the inlet port and wall shear stress 26 at the collection port. A wall shear stress scale is shown at left.

FIG. 16 shows a wall shear stress simulation using Ansys Fluent software with 7E-05 m/s inlet velocity, adjusted range, throughout perfusion compartment 19. The simulation includes wall shear stress 27 at the reaction surface, wall shear stress 28 at first flow barrier, and wall shear stress 29 at the collection compartment. A wall shear stress scale is shown at left.

FIG. 17 shows carbon dioxide steady-state distribution results simulated with 7E-05 m/s inlet velocity of the surface. The simulation includes carbon dioxide (CO₂) concentration at inlet port 30, CO₂ concentration at holding compartment 31, CO₂ concentration at reaction compartment 32, CO₂ concentration at collection compartment 33, and CO₂ concentration at collection port 34, depicted as an inside view projection. A mass fraction of CO₂ scale is shown at left.

### DETAILED DESCRIPTION

The present technology provides methods and a specialized design of perfusion bioreactors that, combined with additional and specialized components, allows for a culture of microbes within the perfusion bioreactors, the synthesis of NPs by those microbes, and the subsequent recovery of the NPs produced. The technology can provide a continuous synthesis of NPs. For example, previous methods of NP production from bacteria include several manual steps of growing the bacteria, plating the cultures, and exchanging the medium (Saklani & Jain, 2013). Any of these steps require human workforce and time, while raising the risk of contamination and leading to reduced efficiency. In the present technology, consistent and adjustable exchange of culture medium is ensured by a perfusion system, which has a structural design that results in a uniform medium exposure to all cultured cells and the prevention of cell loss due to the washout effect, as confirmed by simulation data.

The design can utilize materials with properties that support the adhesion of microbial or bacterial cells throughout the exchange of culture medium and the maintenance of a biologically optimal environment. The design can also satisfy the simultaneous production of multiple elemental types of NPs or NPs with combinations of elements. The technology offers flexibility in utilizing different species of microbes or bacteria, reducing or eliminating manual intervention, and diminishing contamination risks throughout the NP production process. Previous designs that use biological scaffolding materials require the production of such materials and have uneven medium exposure between cells (D. Egger, et al., 2017; J. Schmid, et al., 2018), and they also require the cells to be attached, while they are not easily transferable between different species of bacteria.

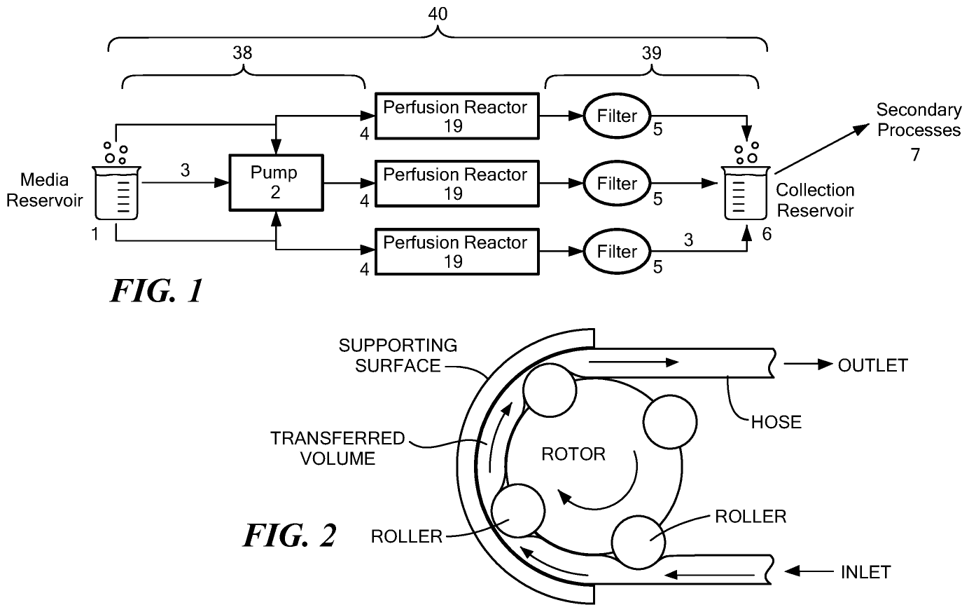

A flow diagram of an exemplary overall process is shown in FIG. 1. Referring to FIG. 1, a perfusion system 40 includes three main phases: the inlet stage 38, the main perfusion reactor(s) 4, and the outlet stage 39. As used herein, “perfusion system” refers to the overall system 40, including the inlet stage 38, the perfusion reactor 4, and the outlet stage 39. Control of NP syntheses can be asserted in all three phases.

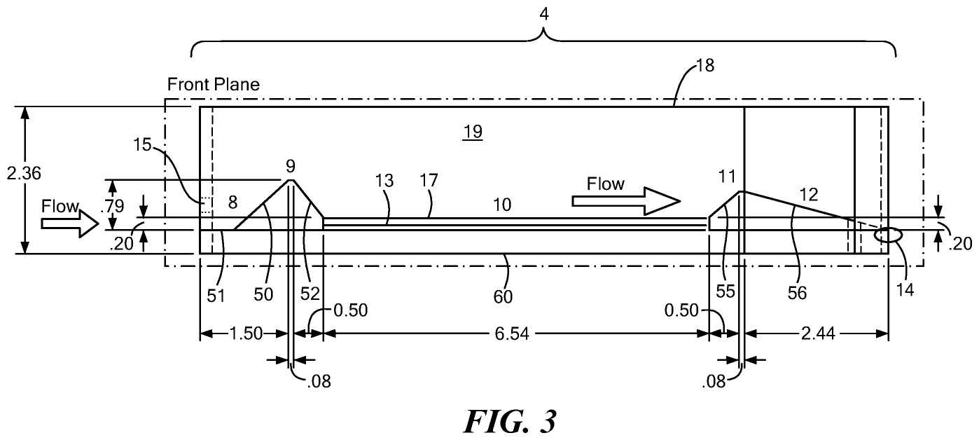

First is the inlet stage 38, where one or more media reservoirs 1 contain prepared media, which is the primary fluid flowing throughout the process. The media reservoirs or reservoir 1, with the media, can support multiple species of NP production. The inlet stage 38 refers to the process upstream to the main perfusion reactor 4. The inlet stage includes the medium reservoir 1, the pump or peristaltic pump 2, and the lines, tubing or flow channels 3, that feed the medium into the perfusion reactor 4. As used herein, “lines” refer to each stream in each tubing or flow channel. Usually, one line refers to one medium stream supplying a specific metal salt solution to a specific species of bacteria. Lines, tubing, or flow channels 3 from the media reservoir 1 can be connected to a multi-channel pump 2 (e.g., a multi-channel peristaltic pump). The perfusion compartment 19, which is described in more detail below, is internal to the main perfusion reactor 4. The flow rate is set depending on the size of each perfusion compartment 19 including a reaction compartment (see 10, FIG. 3) inside each perfusion reactor 4, and the desired medium turnover rate and the thickness of the fluid flow on the surface of the reaction compartment, which is inside the perfusion reactor 4 and perfusion compartment 19. Each line from the pump is connected to the perfusion reactors 4 via tubing, lines, or flow channels 3.

Referring to FIG. 1, the outlet stage 39 starts with a filter 5 from a collection port of the perfusion reactor 4 to collect any washed-out cells or solid waste. The filter 5 is connected to tubes 3, which lead to a collection bottle or collection reservoirs 6. Due to the advantages of the perfusion reactor, this bottle can contain mostly medium and NPs. The collection bottle can either be batched or be connected to further, secondary processing 7 via tubes to form a continuous process. The goal of the secondary processes 7 is to purify the NPs. These processes include, but are not limited to, centrifugation, dialysis, suspension, liquid extraction, freeze-drying, and spray drying (J. D. Robertson, et al., 2016; S. K. Balasubramanian, et al., 2010). There are multiple methods to purify and extract NPs from the medium solution based on available instruments, characteristics of each NP element, and the targeted parameters of applications. Also, it is essential to note that for the design of specific dimensions described in this disclosure, the process can be batched. However, the optimal industrially scaled process can be continuous, with the output from the perfusion reactor acting as the input to the NP extraction and purification.

The medium reservoir or container 1 shown in FIG. 1 contains nutrient solution that helps the bacteria, for example, grow and thrive, along with additional metal salts, acting as input material or reactants to the production of NPs. The medium reservoir can hold a volume of nutrient solution needed to maintain microbial life through a NP synthesis. The medium reservoir containers can be capped with a tubing adaptor such that several tubes can be connected to ensure flow out of the container. The medium inside the reservoir contains nutrient solution, made from one, or a combination of culture medium, differentiation medium, and selection medium, along with any desired metal salt solution. The volume of the medium inside the reservoir can be calculated depending, for example, considering several factors. The number of perfusion reactors or perfusion compartments utilizing the same medium can be considered. The desired medium turnover of the perfusion compartments is considered. Another factor is the volume of the perfusion compartment(s) 19 in the perfusion reactors 4. During medium planning, the total desired time of NP production is also considered.

The volume of the medium reservoir is scalable as long as the time it takes to use up the medium is less than the time it takes for the medium's chemical properties (stability) to be deemed inappropriate for biologics growth. The reservoir containers can be made from, for example, Nalgene, borosilicate glass, acrylic glass, or plastics. The containers can be sturdy, leak-proof, able to withstand the autoclaving process, and remain inactive at biological conditions. In another example, the media reservoirs can be in the form of polymer bags containing sterile medium, and a nozzle configured to attach tubing affixed to the bags.

In FIG. 1, the pump 2 or multi-channel pump can be any suitable pump. FIG. 2 shows an a peristaltic pump, which is preferred. While a peristaltic pump can be utilized for the dimensions of perfusion compartments herein, different types of pumps can be used based on the size of the perfusion compartments 19 in the perfusion reactors 4. Since the perfusion compartments are scalable, the desired flow rate is also scalable, and the pump power and capability are also scalable. Other pump types include centrifugal pumps, axial pumps, and other positive displacement pumps (Pump types, World Pumps, 2003).

The pump can be calibrated initially to determine the specific pressure needed to generate the desired volumetric flow rate. For the design described in some examples, the volumetric flow rate can be small. Therefore, a peristaltic pump can be used to generate this desired flow rate. As used herein, a peristaltic pump refers to a positive displacement pump that utilized a cycle of compression—relaxation to move fluid. A multi-channeled peristaltic pump can be used for the pump 2 in FIG. 1 and can be used to generate the desired flow rate on multiple perfusion compartments simultaneously. A peristaltic pump is a type of pump that utilizes positive displacement to transport the fluid. The pressure generated from the peristaltic pump comes from the constant compression—relaxation cycle that the stream of fluid undergoes.

In FIG. 1, the perfusion compartment 19 is internal to the main perfusion reactor 4 and includes components where the real growth of cells and production of NPs occurs. A side projection, depicting the internal perfusion compartment 19, is shown in FIG. 3. Referring to FIG. 3, the perfusion compartment 19 includes three main volumes: the holding compartment 8, the reaction compartment 10 with a reaction surface 13 and/or an optional removable reaction surface 17, and the collection compartment 12. The collection compartment 12 includes a downhill incline 56 and a collection port 14. The perfusion compartment 19 can include two removable components: the compartment's roof 18, and an optional removable reaction surface or plate 17.

The holding compartment 8 is a space defined as downstream to the inlet medium solution (inlet port 15) and upstream to that of the first flow barrier, or first flow barrier 9. The holding compartment 8 is bounded vertically by the base and the height of the first flow barrier 9. The holding compartment includes a flat surface 51 and an uphill surface (incline) 50, forming the leading surface of the flow barrier 9. The height of the flow barrier 9 and the slope of the uphill incline 50 can be varied as long as the inlet port 15 is vertically inferior to the first flow barrier 9.

The first and second flow barriers are elevated structures extending across the reaction compartment and separating the reaction compartment from the upstream holding compartment and the downstream collection compartment. The first and second flow barriers can be identical or different in structure. In general, each flow barrier will have an upstream uphill incline, a peak, and a downstream downhill incline. The inclines can have any desired profile, including linear or curved, and can be the same or different on each side of a barrier. The peak can be a desired sharp angle, can be rounded, or can include a secondary vertical barrier. Functions of the first barrier can be to evenly distribute flow as well as to reduce flow velocity to a low level that reduces shear stress to a desired low level at the reaction surface. Functions of the second barrier can be to contribute to setting flow and shear stress at the reaction surface and to retain microbial cells in the reaction compartment will allowing nanoparticles to flow across the barrier into the collection compartment.

The holding compartment 8 can serve several functions. The holding compartment can receive inlet medium from the inlet port 15. The holding compartment can prevent excessive stress caused by initial fluid flow from the inlet port. It can prevent non-uniform flow and unequal medium exposure of the bacteria. The holding compartment 8 can provide a space for mixing between medium and metal salt to occur before exposure with bacterial cells in the reaction compartment 10. The holding compartment 8 can provide a buffer zone in case of a system malfunction.

In FIG. 3, the reaction compartment 10 refers to a component of the main perfusion reactor's design. The reaction compartment 10 is placed medially to the overall structure. This compartment refers to the volume that includes the reaction surface 13, the optional removable reaction surface 17. The height of the reaction compartment 10 equals the thickness of the medium flowing through, which is equal to the height of the two flow barriers (see 9 and 11; FIG. 3). The reaction compartment 10 can also be referred to as the reaction volume.

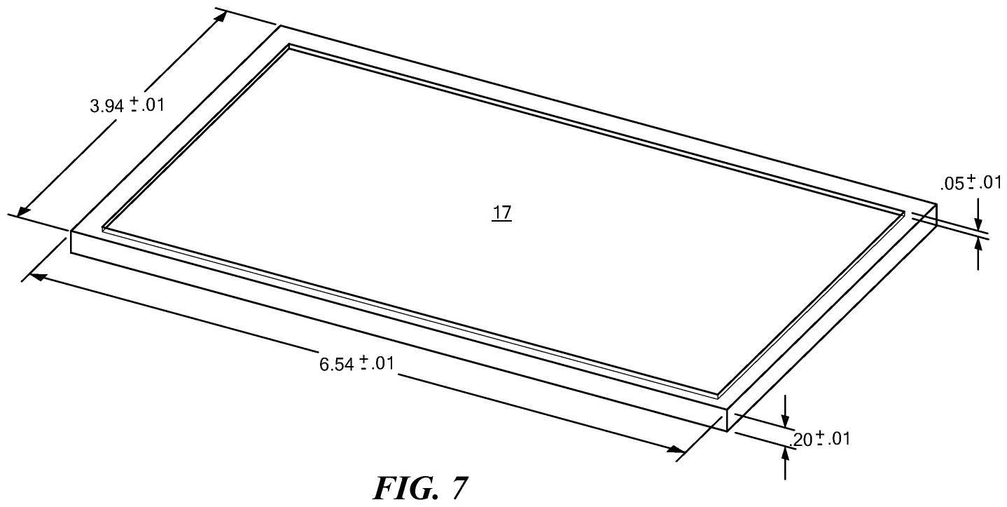

For example, referring to FIG. 3, the reaction compartment 10 is a space defined as downstream to the first flow barrier 9 and upstream to the second flow barrier, or second flow barrier 11. It is bounded vertically by the frame and the height of the two flow barriers. This compartment refers to the control volume in which NPs' production takes place. The reaction compartment includes the downhill incline 52 of the first flow barrier 9, the reaction surface 13 or the optional removable reaction surface 17, and the second flow barrier 11 including the uphill incline 55 of the second flow barrier 11. The reaction compartment 10 contains the reaction surface 13 and/or the optional removable reaction surface 17, bounded by the two ends of the inclines 52 and 55, and the reactor's frame 60 is underneath. This reaction compartment includes the reaction surface 13 where removable reaction surface (or plate) 17 (also see 17, FIG. 7) can be inserted. Optionally, the removable reaction surface (or plate) 17 can be disposed directly upon frame 60. In FIG. 3, the reaction surface 13 or the optional removable reaction surface 17 (plate) is where bacterial cells are plated, grown, and conditioned to produce NPs.

The reaction compartment 10 can serve several functions. The reaction compartment can provide a controlled volume for NPs' production to take place. The reaction compartment can ensure uniform exposure of microbial cells to medium. It can ensure appropriate biological conditions for cell growth. The reaction compartment can prevent cells' washouts and increase the efficiency of NPs produced per area. The reaction compartment can prevent cell washout, for example, by providing a smooth and even perfusion flow with the microbial cells in the reaction compartment 10 along with a barrier including the uphill incline 55 and flow barrier 11 to prevent cell washout.

The goal of the pump is to create a pressure gradient to ensure the constant flow properties of the medium. The pump is a crucial part of the perfusion system, as it controls the volumetric flow rate to the perfusion compartments. Individually, the volumetric flow rate can be calculated using Equation 1.

$$Flow Rate Calculation for Perfusion Compartment$$

$$Q = \frac{{V}_{react}}{t} \qquad Equation 1$$

### In Equation 1:

Q=the volumetric rate that is needed to flow into the perfusion compartment (19, FIG. 3).
Vreact=the volume of the reaction compartment (10, FIG. 3) within the perfusion compartment t=desired medium turnover time.

Referring to FIG. 3, the perfusion compartment 19, or the “board” represents the actual growing and reaction chamber for bacterial (or microbial) cells. The compartment's base 60 can be made out of stainless steel, or other compatible materials, with examples provided in Table 1 below. As used herein, in reference to the base 60: the terms base and frame are used interchangeably. This is the part of the main perfusion reactor's (4, FIG. 1) outer structure. It is responsible for the majority of the perfusion reactor's area footprint. It can be made with stainless steel or other example compatible materials, listed in Table 1.

TABLE 1
Examplary Materials and Specifications for Perfusion Compartment:

| Components | Appropriate Materials | Example Properties |
|---|---|---|
| Base/Frame | Stainless steel, any steel alloy [16], aluminum | Sturdy, anti-corrosive, anti- |
| | and its alloy [17], copper and its alloy [18], | oxidation, unreactive, stability, |
| | titanium and its alloys, acrylic and its derivatives | excellent tensile strength |
| | [19] | |
| Reaction | Polystyrene and its derivatives [20], hydroxyl- | Materials with excellent cell- |
| surface | coated polystyrene, carboxyl-coated polystyrene, | adhesion properties (e.g., |
| (Plate) | cell adhesion protein-coated borosilicate glass | functional group |
| | [21] | intermolecular attraction, |
| | | hydrophilic) |
| Roof | Polymethyl methacrylate (PMMA) [22], | Polymers/Plastics that are |
| | polydimethyl siloxane (PDMS), polyethylene | flexible, and allow gas to |
| | (LDPE and HDPE) [23] | permeate readily |
| Filter | Size: 0.2 um-1 um | Desired NPs have size <100 |
| | Types: PTFE syringe filter, bottle filter, vacuum | nm, washed-out bacteria cell |
| | filter, capsule filter | has a size of 1-2 um |
| Tubing | Diameter: 0.125-12 inches | Tubing/Pipes are scalable |
| Pumps | Peristaltic Pumps, Centrifugal Pumps, Rotary | Pumps and their pressure are |
| | Pumps | scalable |

[16] (Allegheny Ludlum Steel Corporation, Stainless Steel Handbook, 1951)
[17] (Z. Ahmad, 2006)
[18] (J. G. Michael Naboka, 2011)
[19] (JAB J. A. Brydson, Plastics Materials, 1999)
[20] (A. Kozbial, et al., 2018)
[21] (G. M. Edelman, 1983)
[22] (Y. Nakai, et al., 2006)
[22] (Y. Nakai, et al., 2006)
[23] (Budd & Mckeown, 2010)

The perfusion compartment, for example, can be designed using stainless steel as a base, polystyrene as the reaction surface, polymethyl methacrylate as roof, PTFE 0.2 micron filters and PTFE tubes. The perfusion compartment is scalable in each of its described components and all dimensions—both cartesian and cylindrical—unless stated otherwise. Each component of the perfusion compartment can be made with various materials, with examples represented in Table 1. The perfusion compartment itself can be assembled using any combination of components that possess suitable properties of the listed materials. The board's size can be adjusted based on the volume of medium needed for target NP production. The design of the base of this compartment and appropriate simulation models are illustrated in FIG. 3 to FIG. 17.

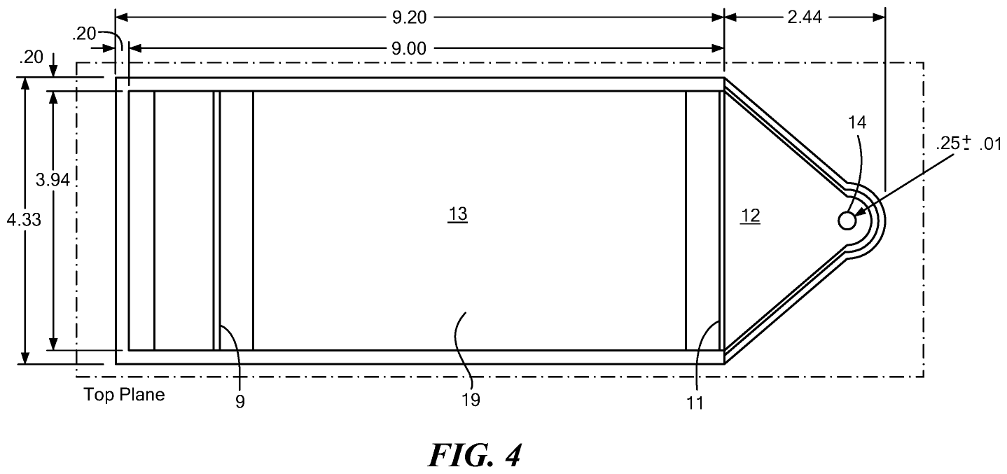

In FIG. 3, the floor of the perfusion compartment 19 is a reaction surface 13, which can optionally be a removable reaction surface 17, and can be made of polystyrene or other compatible materials detailed in Table 1. A top view projection of the perfusion compartment 19 is shown in FIG. 4. In FIG. 4, the reaction surface 13 is depicted without the optional removable reaction surface 17 (FIG. 3). The reaction surface 13 or an optionally removable reaction surface, are surfaces that microbial cells are plated on. FIG. 7 shows the separated or removable reaction surface 17, where cells are seeded and adhered to, with dimensions in inches. The reaction surface is where the microbes grow and where the production of NPs occurs.

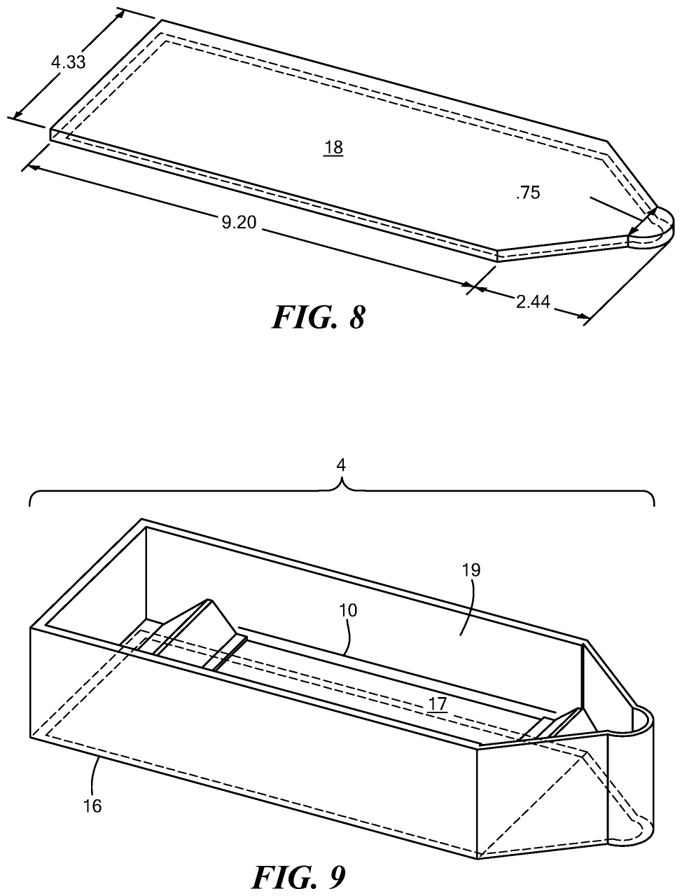

The roof (18, FIG. 3) refers to the top of the perfusion compartment. For example, the roof refers to the apparatus that is placed on top of the frame, and an example roof 18 is shown in FIG. 8. The compartment's roof can be made with polymethyl methacrylate (PMMA), or a gas-permeable comparable polymer, detailed in Table 1.

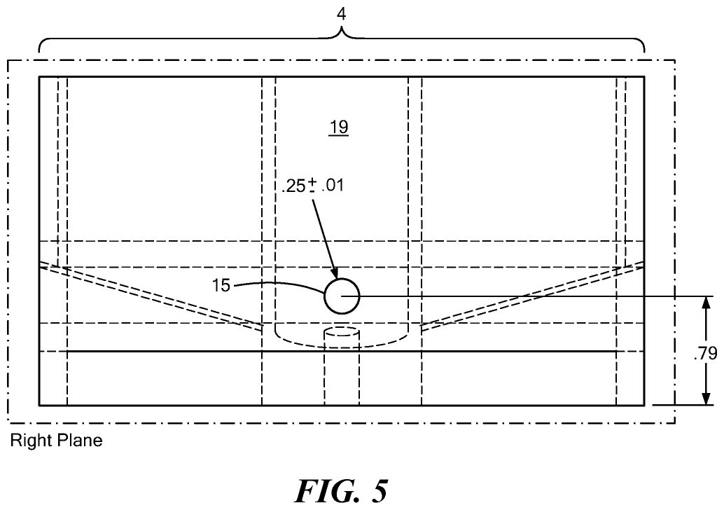

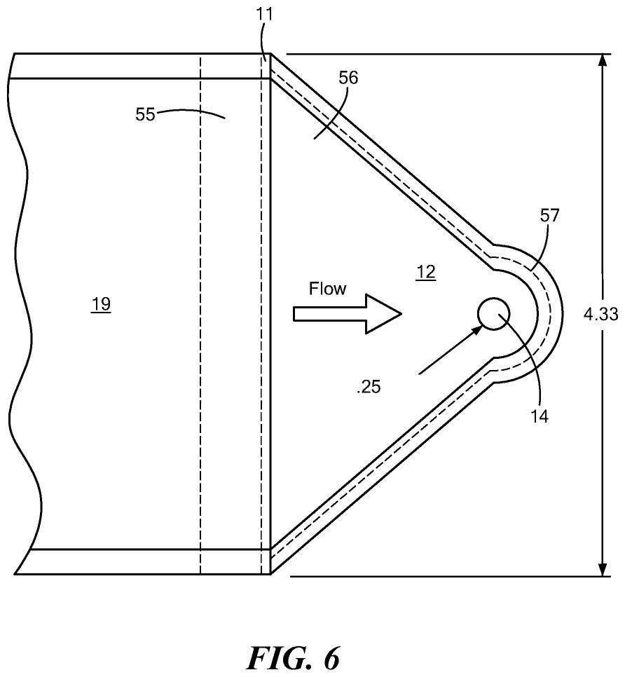

FIG. 5 shows a right view projection of the perfusion compartment 19 with an inlet port 15. FIG. 6 shows a bottom view projection of the perfusion compartment 19, zoomed in at the collection compartment 12 showing the collection port 14.

As shown in FIG. 3, the collection compartment 12 includes a downhill incline 56. As used herein, the downhill incline 56 is a component of the main perfusion reactor's design. As illustrated in FIG. 6, the downhill incline 56 placed downstream of the NPs production site, and upstream to the collection port 14. It is designed as an incline to reduce the fluid's stress on the tubing and keep the flow uniform and laminar throughout the reactor.

In FIG. 6, the reacted medium, that contains NPs, flows to the collection port 14, which resides at the bottom end of the collection compartment 12, leading to further downstream processing. The collection compartment 12 is a space defined as downstream to the second flow barrier 11 and upstream of the collection port 14. The collection compartment 12 is bounded vertically by the frame and the height of the second flow barrier 11. The collection compartment 12 refers to the control volume in which the medium is saturated with NPs after production occurs. The collection compartment 12 includes a downhill surface 56 and an orifice that allows the stream to flow out of the perfusion reaction called the collection port 14. The collection compartment 12 has an equilaterally triangular shape, converging at a circular end 57 that is concentric to the collection port. As illustrated in FIG. 6, the size of each triangular leg of the collection compartment 12 and the radius of the concentric convergence 57 around the collection port 14, are scalable, as long as the radius is not more significant than the horizontal distance between the collection port to the second flow barrier (11, FIG. 3). The collection compartment 12 can serve several functions. The collection compartment can provide a space of transition from production of NPs to collection of NPs. The collection compartment can provide a buffer volume in case of a system malfunction. The collection compartment can ensure consistent outflow of the medium. The collection compartment can reduce shear stress in the outlet tubing.

The collection port 14 is a space defined as downstream to the collection compartment 12. The collection port 14 is an orifice space with a circle base extruded through the frame of the perfusion compartment, and an example is depicted in FIG. 4 and FIG. 6. This orifice 14 is concentric to the convergent circular shape 57 at the end of the collection compartment 12. The bottom of this orifice is a flat surface, while the top of this orifice can be vertically uneven due to the downhill surface 56. The inside surface area of the orifice can be threaded using a tap of the appropriate size to be connected to tubing for outlet. The collection port size is scalable, as long as a thread size and tubes can be obtained.

The collection port 14 is a component of the main perfusion reactor's design. It is placed downstream of the NPs production site, and this is the port where the NPs-medium solution flows out of the reactor. It is located at the end of the reactor.

The outlet stage refers to the process downstream to the main perfusion reactor. The outlet stage includes the tubing, the filters (see item 5, FIG. 1), collection bottles, and coupling secondary processes (see item 7, FIG. 1).

The filter and collection systems are located downstream from the perfusion compartment. The filter accepts the flow from the collection port. The filter system's goal is to eliminate particles that have the size greater than the desired NP sizes. These eliminated particles include washed out cells, cell metabolites, or other unknown metabolic wastes. Desired NPs have a size of less than about 100 nm (0.1 μm). Therefore, the filter membrane pore size can be chosen to be about 0.2 μm for the described compartment. An example filter for the described compartment can be chosen to be a polytetrafluoroethylene (PTFE) syringe filter, connected to inlet and outlet tubes using Luer locks. However, filters with different membrane sizes and materials can be used, and an example list of appropriate filters is presented in Table 1. Multiple filters in series can be installed in the system.

The collection bottles (or containers) are located downstream from the filters. The collection bottles (see 6; FIG. 1) refers to the container that holds the volume of metabolized medium and produced NPs. This apparatus is placed after the filtering process (see item 5, FIG. 1). The collection bottles can be capped with a tubing adaptor such that several tubings can be connected to ensure flow into and out of the container. The solution inside the reservoir contains a metabolized solution, along with the suspended NPs.

The volume of the bottles is scalable. The collection bottles can be made from, for example, Nalgene, borosilicate glass, acrylic glass, or plastics, as long as the bottles are sturdy, leak-proof, able to withstand the autoclaving process and remains inactive at biological conditions. In another example, collection can be in a flexible polymer reservoir.

The collected bottles can be removed from the system and transferred as the inlet to secondary processes, making the whole system a batch or semi-batch system. The collection bottles can also have an outlet flow or be coupled into secondary processes, making the entire system continuous. These processes include, but are not limited to, centrifugation, dialysis, suspension, liquid extraction, freeze-drying, and spray drying. The goals of the secondary processes are the separation and purification of produced NPs.

In FIG. 8, the roof 18, of the perfusion compartment can be made with the same horizontal dimension as the device's frame. Due to the need to maintain sterility while also ensure sufficient molecular transport with the incubator's environment, the roof can be made from gas-permeable solid, such as polymethyl methacrylate (PMMA), or other appropriate materials listed in Table 1. The thickness of the roof should be less than or equivalent to that of the thickness of the frame's vertical surfaces.

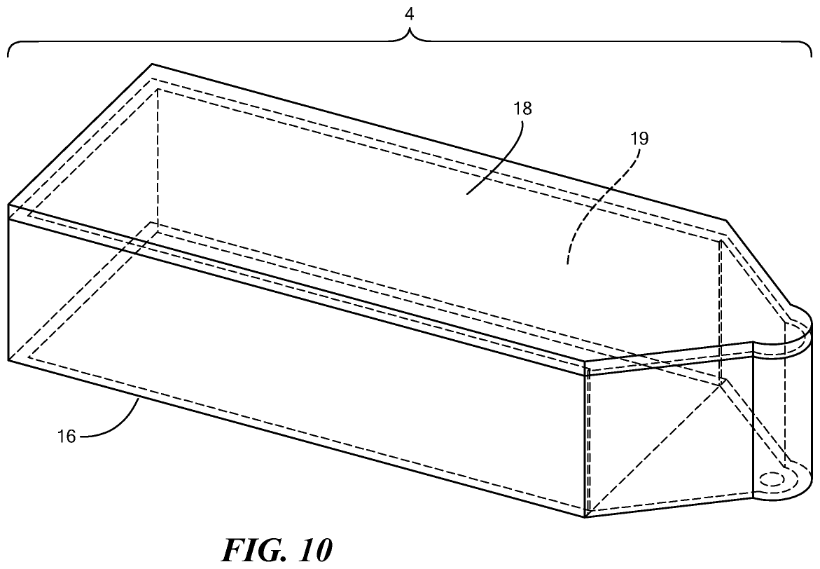

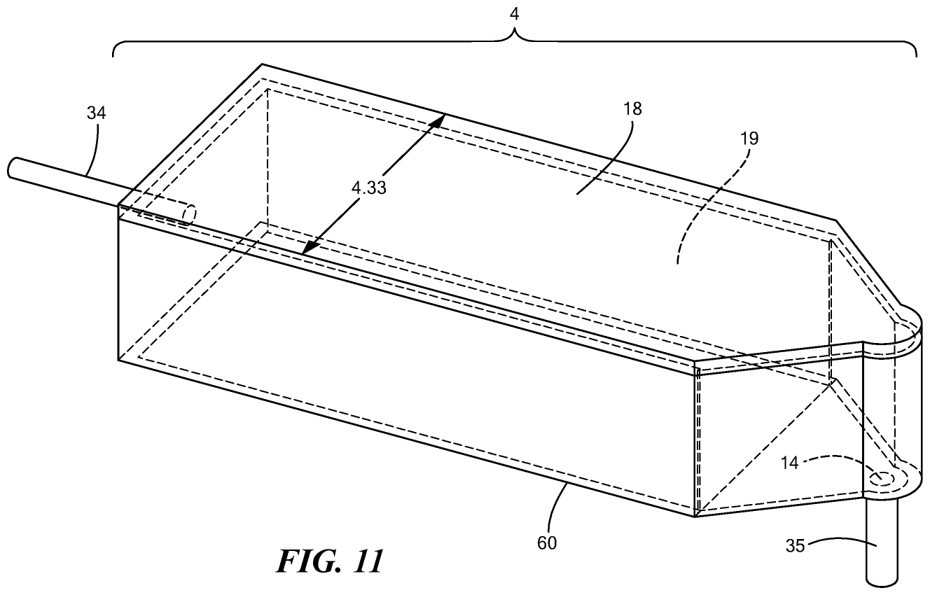

FIG. 9 depicts a perfusion compartment 19 including an assembled frame 16 (grey) and a reaction surface (white) 17 but without the roof 18, which is depicted in FIG. 8. The removable reaction surface 17 is positioned within the reaction compartment 10. In FIG. 10, the assembled frame 16 (dark grey) and roof 18 (light grey) enclose the perfusion compartment 19. FIG. 11 depicts the main perfusion reactor 4 appearance from the outside, which includes the frame 16, and roof 18. The inlet 34, and outlet 35 tubing are also depicted.

An objective of the technology is to increase the workflow of the NP production process, using synthesis from bacterial or other microbial cells. This technology can automate the production of NPs through a semi-continuous perfusion system in which medium can be exchanged in a sterile and constant manner. The technology can provide an increase in efficiency and quality of the NP product and a decrease in contamination risk. The perfusion system and methods herein can fulfill, for example, a constant supply of nutrient medium at a desired rate. Constant removal of metabolized medium at desired rate can be achieved. Uniform and appropriate medium exposure to all seeded cells is provided. Uniform and consistent maintenance of the appropriate biological environment can be achieved. Prevention of cell washout due to perfusion is enabled through the design and methods herein. Appropriate size filtration before NPs collection can be implemented. The technology enables the ability to operate multiple perfusion reactors simultaneously. The system and methods provide the ability to customize based on bacteria or other microbial cells and metal element's characteristics. Minimization of manual intervention is enabled throughout NPs synthesis.

Multiple perfusion compartment simulations demonstrate how the features of the technology can be implemented. The simulation results for the designed perfusion reactor indicate that medium exchange occurs at a constant and adjustable rate. The flow results in uniform medium exposure to all cells seeded on the reaction surface, except the area near the vertical wall. The flow is fastest at the inlet and outlet port, as expected. The design successfully prevents an uneven distribution of flow velocity that would otherwise occur. The design also assists the outflow of medium, ensuring all metabolized medium are removed from the compartment. Simulations also indicate that the wall stress on the reaction surface is negligibly small, limiting possible cell washout. Besides, simulations indicate that given the appropriate incubator parameters, the perfusion reactor maintains a biologically appropriate environment at a steady-state (Yang & Xiong, 2012).

The perfusion compartment is used to ensure the steady, consistent, and clean generation of biogenic NPs by utilizing mature bacterial cell enzymatic activities. Some exemplary objectives that are enabled for the system involved are: exchange of medium over the desired period (medium turnover time), avoidance of cell washout and cell loss, continuous perfusion throughout the growing period for microbes or bacteria. The system can maintain biocompatible conditions (e.g., temperature, pressure, carbon dioxide concentration), can reduce manual labor to reduce consumed resources and prevent errors, and can maximize efficiency and throughput.

Some of these objectives are simulated to provide proof of concept to the viability of the prototype. Ansys Fluent, a fluid transportive simulation program, is used to generate these results. Ansys Fluent utilizes built-in physics in conservation and transport principles of momentum, phase, materials, species, and energy to demonstrate different properties throughout the flow within the specified geometry.

Based on the control volume of the perfusion compartment's reaction surface, along with the area of the inlet port, the kinetic velocity of the medium can be estimated to be 7.0E-05 m/s for all simulations. However, the kinetic velocity of the medium can be scalable as long as the resulting flow through the flat plate remains laminar. Flow over a flat plate can be assumed to be laminar if its Reynolds number does not exceed 5.0E05 m/s. Reynold's number can be calculated using Equation 2 (Wesenl & Bird, 2006).

$$Reynold ’ s Number formula for flow over a flat plate$$

$$Re = \frac{ρ * \vec{v} * x}{μ} \qquad Equation 2$$

$$ρ = Density of the fluid of interest$$

$$\vec{v} = velocity vector of the fluid$$

$$x = length over which the fluid flow$$

$$μ = viscosity of the fluid of interest$$

The simulation of the perfusion compartment utilizes several assumptions. The medium's properties are assumed to be similar to that of water because concentration of water in the solution is significantly higher than other components. A no-slip condition at a wall is assumed—common in microfluidic simulations (B. E. Rapp, 2016). Constant intensive properties are assumed—change in temperature and pressure is negligibly small. Steady State—system at equilibrium is assumed. Finally, it is assumed no mass flux at the reaction surface—low diffusion coefficient of steel.

These assumptions are applied to constitutive equations that are principles of momentum, energy, and mass transfers. For the simulations, surfaces are made to investigate the flow profile of the device. Surface A is a cross-sectional surface at y=30 mm from the bottom of the device. Surface B is a cross-sectional surface at x=200 mm from the left of the device. Surface C is a cross-sectional surface at midline width z of the device.

Fluid flow simulations are performed to provide proof of concept of the illustration of the medium perfusion throughout the compartment. Fluid flow simulation is used to determine the area of highest medium exposure and to identify a stagnant area that does not ensure consistent medium turnover. The constitutive equation that the model is based on includes the continuity equation (Equation 3) and the three-dimensional Navier-Stokes equation (Equation 4), (Wesenl & Bird, 2006).

$$Continuity Equation$$

$$∇ \cdot \vec{V} = 0 \qquad Equation 3$$

$$Navier - Stokes Equation$$

$$ρ \frac{D \vec{V}}{Dt} = - ∇ P + ρ g + μ {∇}^{2} \vec{V} \qquad Equation 4$$

$$∇ \cdot \vec{V} = gradient of velocity vector$$

$$∇ P = gradient of pressure$$

$$ρ = density of the fluid$$

$$\frac{D \vec{V}}{Dt} = total derivative of velocity vector$$

$$g = gravity acceleration$$

$$μ = viscosity of the fluid$$

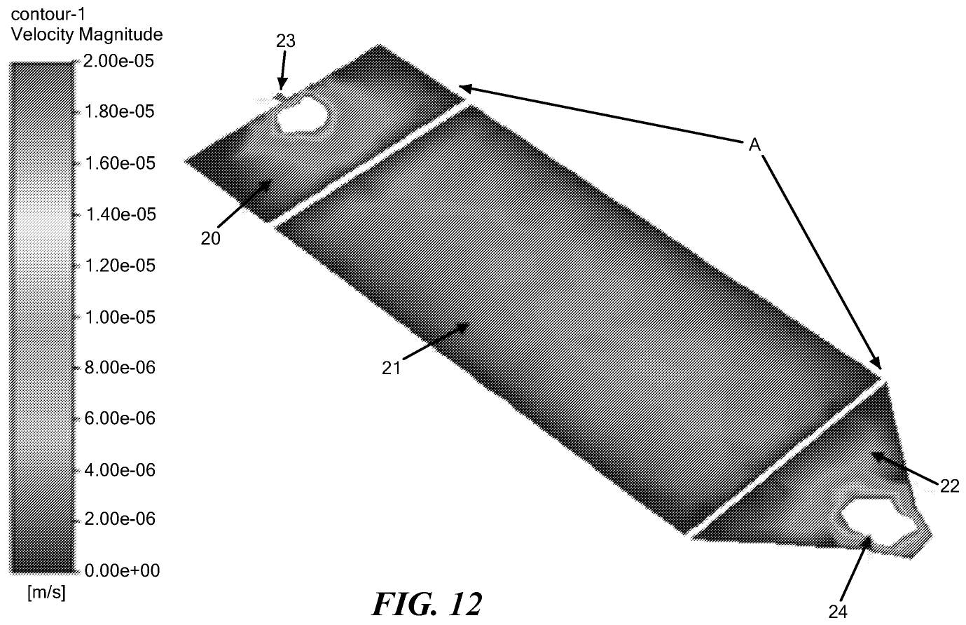

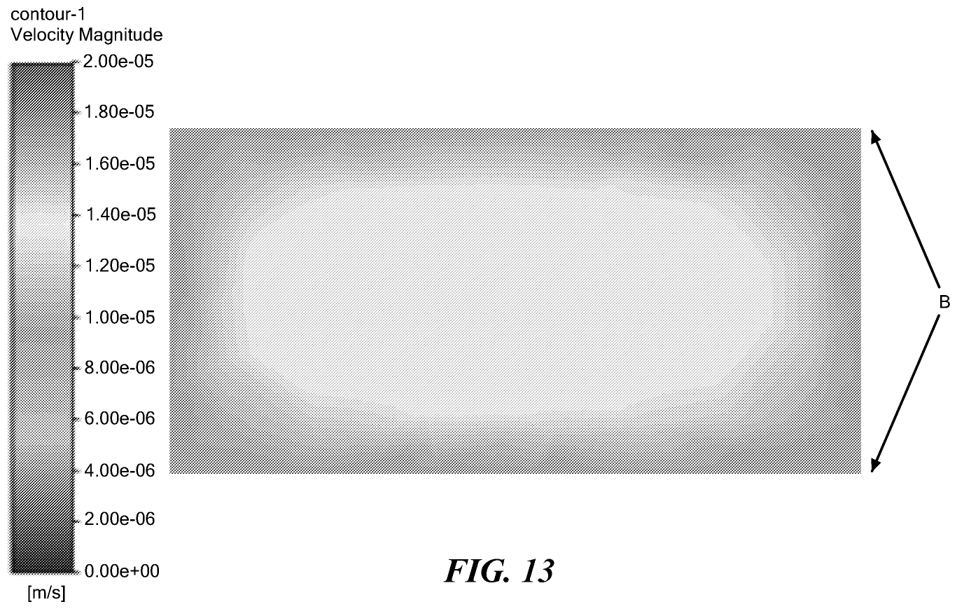

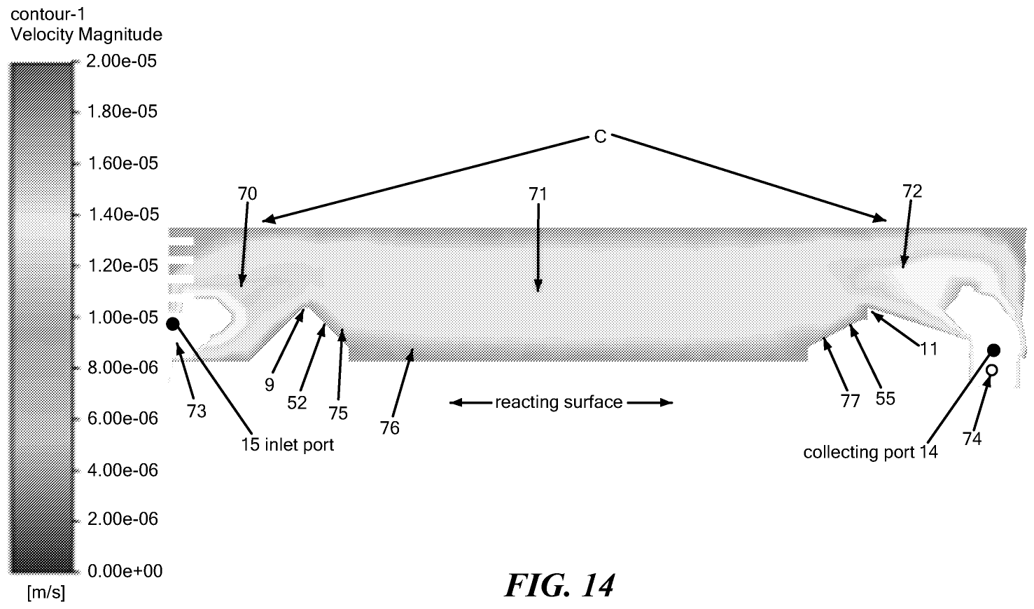

The results of the fluid flow simulations are presented in FIG. 12, FIG. 13, and FIG. 14. In FIG. 12, fluid simulation results with 7E-05 m/s inlet velocity of the surface A parallel to the reaction surface are presented. FIG. 12 includes (20) velocity magnitude at holding compartment, (21) velocity magnitude at reaction compartment, (22) velocity magnitude at collection compartment, (23) velocity magnitude at inlet port, and (24) velocity magnitude at collection port, in isometric view.

FIG. 13 shows fluid simulation results with 7E-05 m/s inlet velocity of surface B parallel to the inlet port and perpendicular to the reaction surface and surface A in right view projection.

FIG. 14 shows fluid simulation results with 7E-05 m/s inlet velocity of surface C perpendicular to the reaction surface and the inlet port. In FIG. 14 are shown (70) velocity magnitude at holding compartment, (71) velocity magnitude at reaction compartment, (72) velocity magnitude at collection compartment, (73) velocity magnitude at inlet port, (74) velocity magnitude at collection port, (75) velocity magnitude at 52 downhill incline of 9 first flow barrier, (76) velocity magnitude at reaction surface, and (77) velocity magnitude at 55 uphill incline of the 11 second flow barrier, inside view projections.

The fluid flow results through the perfusion compartment indicate that the flow displays uniform and symmetrical pattern within the reaction compartment and the velocity reaches its maximum at the inlet and collection port. The fluid flow results through the perfusion compartment indicate that the area near the walls in all directions receives poorer fluid flow exposure, as expected from the no-slip condition. A low-velocity magnitude throughout the perfusion compartment, as expected from small inlet velocity of 7E-05 m/s, is indicated. The fluid flow results through the perfusion compartment indicate the holding compartment prevents the initial spike in velocity from the inlet port, thus ensuring consistent and creeping medium exposure in the reaction compartment.

Wall stress studies are also conducted. It is crucial to examine the wall shear stress to ensure that any material used in building the perfusion compartment can withstand the flow over a long time. Besides, the examination of wall stress can confirm the prevention of the washout of cells. Low, uniform wall stress over the reaction surface indicates a less likely chance of bacteria cells getting carried by the medium's flow over the surface.

It is hypothesized that the shear stress would remain low throughout the perfusion compartment and consistent throughout the reaction compartment due to the low-velocity flow rate of the solution at a steady state. The wall shear stress in the model is calculated using Newton's Law of Viscosity (Equation 5) (Wesenl & Bird, 2006).

$$Newton ’ s Law of Viscosity of flow in x - direction acting on y - surface$$

$${τ}_{yx} = μ \frac{{dv}_{x}}{dy} \qquad Equation 5$$

$${τ}_{yx} = wall shear in x - direction flow acting on y - direction surface$$

$${v}_{x} = velocity vector in x - direction$$

$$μ = viscosity of the fluid$$

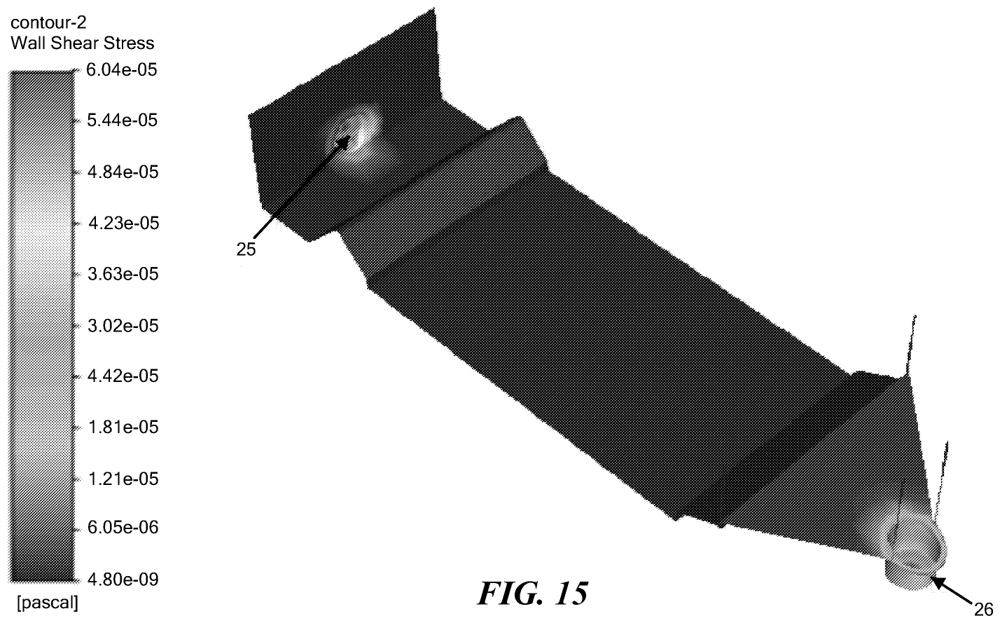

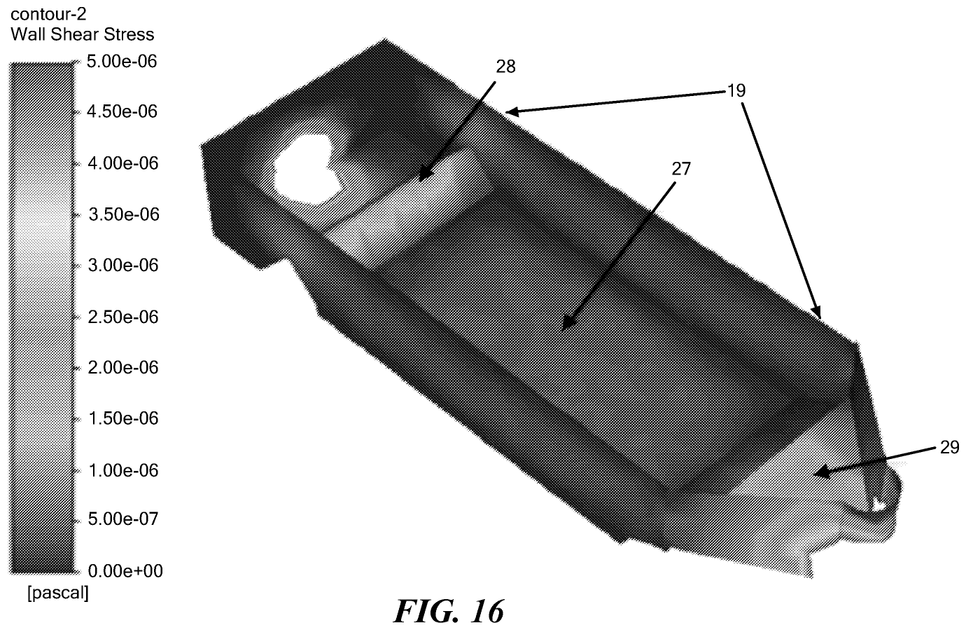

The results of the wall shear stress simulations are presented in FIG. 15 and FIG. 16. FIG. shows the wall shear stress simulation using Ansys Fluent software with 7E-05 m/s inlet velocity at the surface parallel to the reaction surface. The simulation includes (25) wall shear stress at the inlet port and (26) wall shear stress at the collection port. The simulation indicates that the shear stress at the inlet (25, FIG. 15) and collection port (26, FIG. 15) are significantly higher than those at any other surface of the compartment. This result confirms the expectation since at inlet port and collection port; the velocity is its highest. However, the shear stress is still minuscule, with a maximum of approximately 4E-05 Pascals at both ports.

FIG. 16 shows the wall shear stress simulation using Ansys Fluent software with 7E-05 m/s inlet velocity, adjusted range, throughout the (19) perfusion compartment. The simulation includes (27) wall shear stress at the reaction surface, (28) wall shear stress at first flow barrier, and (29) wall shear stress at the collection compartment. FIG. 16 displays the result of the same simulation, but the contour's range is adjusted to illustrate the distribution of stress over the compartment. The inlet port and collection port has no contour because their shear stresses are out of the adjusted range. The result shows the shear stress at the reaction surface, the first flow barrier (28, FIG. 16), and the collection compartment (29, FIG. 16) downstream to the second flow barrier.

The wall shear stress result through the perfusion compartment indicates that there is a low wall shear stress across the device, as predicted due to low fluid flow velocity. Maximum stress at the inlet is about 6E-05 Pascals, outlet about 3E-05 Pascals. The wall shear stress result through the perfusion compartment indicates within the device, shear stress is higher close to the inlet port and every uphill and downhill section. Maximum is at the top of the two hills, with a magnitude of 2E-06 Pascals. The wall shear stress result through the perfusion compartment indicates a low and uniform shear stress at the reaction surface, ensuring maximum cell retention in the compartment.

Carbon dioxide distribution within the perfusion compartment was studied, for example, to demonstrate viable conditions for culture. It is important to maintain a biologically compatible environment at the reaction surface. Since the device's roof is made using PMMA polymer layer, or other appropriate material listed in Table 1, which maintains gas permeability property, the environment in the device can be assumed to contain 5% Carbon Dioxide if the whole device is placed in a CO₂ incubator. The carbon dioxide distribution can be calculated throughout the liquid film flowing using mass transport constitutive principles. The following assumptions are applied to the simulation: 1) Diffusion only (no convective mass transport), 2) Constant mass diffusion coefficient, 3) Constant fluid properties (Newtonian fluid), 4) Steady state (non-transient), 5) No mass flux at any wall of the device (boundary condition at the solid-liquid surface), 6) 5% Carbon dioxide incubator environment (boundary condition at the liquid-gas surface). The simulation is based on the diffusion-only mass transport constitutive principle, written in Equation 6, called Fick's second law of diffusion (Wesenl & Bird, 2006).

$$Fick ’ s second law of diffusion$$

$$\frac{\partial {c}_{A}}{\partial t} = {D}_{mix} [ \frac{{\partial }^{2} {c}_{A}}{\partial {x}^{2}} + \frac{{\partial }^{2} {c}_{A}}{\partial {y}^{2}} + \frac{{\partial }^{2} {c}_{A}}{\partial {z}^{2}} ] \qquad Equation 6$$

$${c}_{A} = concentration of carbon dioxide$$

$${D}_{mix} = diffusion coefficient between carbon dioxide and water$$

$$t = time$$

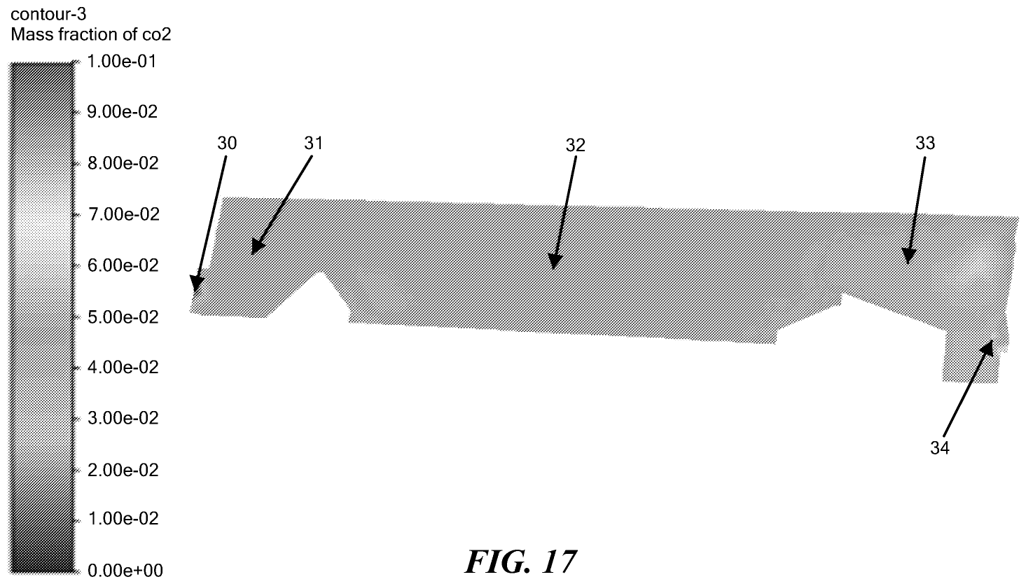

Using these assumptions, the reaction surface carbon dioxide distribution at steady state in a CO₂ incubator is demonstrated in FIG. 17. FIG. 17 shows carbon dioxide steady-state distribution results simulated with 7E-05 m/s inlet velocity of the surface. The simulation includes (30) carbon dioxide (CO₂) concentration at inlet port, (31) CO₂ concentration at holding compartment, (32) CO₂ concentration at reaction compartment, (33) CO₂ concentration at collection compartment, and (34) CO₂ concentration at collection port, all depicted as an inside view projection. The result indicates that the carbon dioxide concentration at steady state throughout the compartment is uniform and approaching 5%—the level appropriate for bacterial life. Using a gas permeable material for the roof of the compartment allows the incubator's air to diffuse into the device, negating the need to infuse carbon dioxide gas into the medium.

The technology disclosed herein presents methods and devices that allow the continuous perfusion of nutrient medium to seeded bacterial cells for NP production. The detailed examples refer to the perfusion system consisting of three stages: inlet stage, perfusion compartment, and outlet stage. The inlet stage involves the medium reservoir and the peristaltic pump, which provide flow to multiple parallel lines of production. The outlet stage includes a series of filters and a collection bottle connected to secondary purification processes. The perfusion compartment is designed as a rectangular body, combined with an adjacent triangular portion. The perfusion compartment is divided into a holding compartment, a reaction compartment, and a collection compartment.

The system design ensures efficient conditions for bacterial growth, and therefore, encourages the NP production from bacterial or other microbial cells. The described examples allow constant supply of new medium and constant removal of medium at the desired rate. The designed perfusion compartment is shown to allow uniform and consistent medium exposure to seeded cells on the reaction surface and uniform and consistent maintenance of target biological conditions. The flow simulations within the perfusion reaction also indicates minimal wall shear stress on the reaction surface, which limits the potential of cell washout. This feature ensures constant and optimal cell density on the reaction surface, allowing the most efficient bacterial growth and NP production.

The embodiments herein enable useful and versatile production of NP using bacterial cells. The removable reaction surface can allow fast and flexible production of different elemental NP using different species of bacteria. The peristaltic pump allows multiple perfusion reactors to be operated at the same time. The series of filters ensure only the appropriate size of NP are collected. The connection from the collection bottle to further purification processes supports potential automated and continuous production of NPs. The closed system minimizes manual intervention, which reduces the chance of contamination and other procedural errors.

The bioreactor design presents a consistent and adjustable exchange of culture medium, which is ensured by the perfusion system, whose structural design results in the uniform medium exposure to all cultured cells and the prevention of cell loss due to the washout effect, as confirmed by simulation data. The design can utilize material whose properties support the adhesion of bacterial cells throughout the exchange of culture medium and the maintenance of a biologically optimal environment. The design also satisfies the simultaneous production of multiple elemental types of NPs. The design offers flexibility in utilizing multiple species of bacteria, reducing manual intervention, and diminishing contamination risks throughout the NP production process. The devices and methods can produce a suspension of nanoparticles that is essentially free of the cells utilized to synthesize the nanoparticles. As used herein, the term “essentially free” refers to a suspension that is less than 5% (weight/weight), less than 2% (weight/weight), less than 1% (weight/weight), less than 0.5% (weight/weight), less than 0.2% (weight/weight), or less than 0.1% (weight/weight) cells. The measurement is conducted by measuring the weight of the cells present and comparing to total weight of the suspension.

Examples of novel and unusual features of the technology include: 1) a division of conventional flat-bed reactor into a holding compartment, a reaction compartment and a collection compartment, 2) a removable reaction surface that enhance cell adherence, 3) an inline filtering system downstream of the reactor to purify NP product, 4) a gas-permeable roof of the reactor to ensure appropriate biologic environment.

Examples of advantages and improvements include: 1) consistent and uniform exposure of cells on reaction surface to the flowing medium, 2) limit the washout of cells from the perfusion system, increasing efficiency and saving resources, 3) multiple production lines simultaneously, and 4) adaptable to different production of various NP's elements from various bacteria species due to removable reaction surface.

Different types of cells have capability to produce metallic NPs and can be used in the present technology, including microbial cells and mammalian cells, including stem cells. Different metal elements and their oxides or sulfides can be constituents of NPs made according to the present technology. The synthesized NPs can be amorphous, crystalline, co-crystalline, metallic, glass, or a combination thereof. Examples of various bacteria suitable for NP production and suitable elements are shown in Table 2.

TABLE 2
Examples of Bacteria for NP Synthesis

| | Nanoparticle's | | |
|---|---|---|---|
| Microorganism | Elements | Localization/morphology | Size and shape |
| Bacillus subtilis 168 | Au | Inside cell wall | 5-25 nm, octahedral |
| Aquaspirillum | Fe3O4 | Intracellular | 40-50 nm, octahedral |
| magnetotacticum | | | prism |
| Klebsiella pneumoniae | CdS | Cell surface | 5-200 nm |
| Pseudomonas stutzeri | Ag, Ag2S | Periplasmic space | <200 nm, nano-crystals |
| AG259 | | | |
| Lactobacillus sp. | Au, Ag, Au—Ag | Intracellular | 20-50 nm, |
| | | | hexagonal/contour |
| Desulfovibrio | Pd | Cell surface | ~50 nm |
| desulfuricans | | | |
| Corynebacterium sp. | Ag | Cell wall | 10-15 nm |
| SH09 | | | |
| P. boryanum UTEX | Pt | Intracellular | 30-0.3 nm, spherical, |
| 485 | | | chains, dendritic |
| Lactobacillus sp. | Ti | Intracellular | 40-60 nm, Spherical |

(from Das, et al., 2017).

Examples of various fungi cells suitable for NP production and suitable elements are shown in Table 3.

TABLE 3
Examples of Fungi for NP Synthesis

| | Nanoparticle's | | |
|---|---|---|---|
| Fungal Species | Elements | Localization/morphology | Size and shape |
| Fusarium oxysporum | Au | Extracellular | 20-40 nm spherical, |
| | | | triangular |
| F. oxysporum | Zr | Extracellular | 3-11 nm quasi- |
| | | | spherical |
| F. oxysporum | Au—Ag | Extracellular | 8-14 nm |
| F. oxysporum | Si, | Extracellular | 5-15 nm, quasi- |
| | | | spherical |
| F. oxysporum | Ti | Extracellular | 6-13 nm, spherical |
| F. oxysporum | Pt | Extracellular | 10-50 triangle, |
| | | | hexagons, square, |
| | | | rectangles |
| F. oxysporum | BaTiO₃ | Extracellular | 4 nm |
| V. luteoalbum and | Au | Extracellular | <10 nm spherical |
| isolate 6-3 | | | |
| Aspergillus flavus | Ag | Extracellular | 8.9 nm |
| Coriolus versicolor | Ag | Intracellular and | 25-75 nm, 444-491 |
| | | extracellular | nm spherical |

(from Das, et al., 2017).

The technology can provide, for example, an advantage of combining the cell culture and cell production, collection and preliminary purification of NPs into one equipment. The technology can be provided as a kit comprising any of the examples or features discussed above.

### EXAMPLES

### Example 1: Fluid Flow Simulations

For the simulations, surfaces were made to investigate the flow profile of the device. Surface A: cross-sectional surface at y=30 mm from the bottom of the device (e.g., FIG. 12). Surface B: cross-sectional surface at x=200 mm from the left of the device (e.g., FIG. 13). Surface C: cross-sectional surface at midline width z of the device (e.g., FIG. 14).

Dimensions in FIG. 3 were converted to centimeters in Table 4 below:

TABLE 4
Example Length Dimensions FIG. 3:
FIG. 3 Lengths

| Item: | Item Number: | Inches: | Centimeters: |
|---|---|---|---|
| holding compartment | 8 | 1.50 | 3.81 |
| first flow barrier | 9 | 0.08 | 0.20 |
| downhill incline of first flow | 52 | 0.50 | 1.27 |
| barrier | | | |
| reaction compartment | 10 | 6.54 | 16.61 |
| uphill incline of second flow | 55 | 0.50 | 1.27 |
| barrier | | | |
| second flow barrier | 11 | 0.08 | 0.20 |

The fluid flow simulations were performed to provide proof of concept of the illustration of the medium perfusion throughout the compartment. It was used to determine the area of highest medium exposure and to identify a stagnant area that does not ensure consistent medium turnover. The constitutive equation that the model was based on includes the continuity equation (Equation 3) and the three-dimensional Navier-Stokes equation (Equation 4) (Wesenl & Bird, 2006).

$$Continuity Equation$$

$$∇ \cdot \vec{V} = 0 \qquad Equation 3$$

$$Navier - Stokes Equation$$

$$ρ \frac{D \vec{V}}{Dt} = - ∇ P + ρ g + μ {∇}^{2} \vec{V} \qquad Equation 4$$

$$∇ \cdot \vec{V} = gradient of velocity vector$$

$$∇ P = gradient of pressure$$

$$ρ = density of the fluid$$

$$\frac{D \vec{V}}{Dt} = total derivative of velocity vector$$

$$g = gravity acceleration$$

$$μ = viscosity of the fluid$$

The results of the fluid flow simulations are presented in FIG. 12, FIG. 13, and FIG. 14. In FIG. 12, fluid simulation results with 7E-05 m/s inlet velocity of the surface A parallel to the reaction surface are presented. FIG. 12 includes (20) velocity magnitude at holding compartment, (21) velocity magnitude at reaction compartment, (22) velocity magnitude at collection compartment, (23) velocity magnitude at inlet port, and (24) velocity magnitude at collection port in isometric view.

FIG. 13 shows fluid simulation results with 7E-05 m/s inlet velocity of surface B parallel to the inlet port and perpendicular to the reaction surface and surface A in right view projection.

FIG. 14 shows fluid simulation results with 7E-05 m/s inlet velocity of surface C perpendicular to the reaction surface and the inlet port. In FIG. 14 are shown (70) velocity magnitude at holding compartment, (71) velocity magnitude at reaction compartment, (72) velocity magnitude at collection compartment, (73) velocity magnitude at inlet port, (74) velocity magnitude at collection port, (75) velocity magnitude at 52 downhill incline of 9 first flow barrier, (76) velocity magnitude at reaction surface, and (77) velocity magnitude at 55 uphill incline of the 11 second flow barrier, inside view projections.

The fluid flow results through the perfusion compartment indicated that the flow displays uniform and symmetrical pattern within the reaction compartment. The fluid flow results through the perfusion compartment indicated that the velocity reaches its maximum at the inlet and collection port. The fluid flow results through the perfusion compartment indicated that the area near the walls in all directions receives poorer fluid flow exposure, as expected from the no-slip condition. The fluid flow results through the perfusion compartment indicated that there is low-velocity magnitude throughout the perfusion compartment, as expected from small inlet velocity of 7E-05 m/s. The fluid flow results through the perfusion compartment indicated that the holding compartment prevents the initial spike in velocity from the inlet port, thus ensuring consistent and creeping medium exposure in the reaction compartment.

### Example 2: Wall Stress Studies

Wall stress studies were conducted. It was crucial to examine the wall shear stress to ensure that any material used in building the perfusion compartment can withstand the flow over a long time. The examination of wall stress can confirm the prevention of the washout of cells. Low, uniform wall stress over the reaction surface indicates a less likely chance of bacteria cells getting carried by the medium's flow over the surface.

It was hypothesized that the shear stress would remain low throughout the perfusion compartment and consistent throughout the reaction compartment due to the low-velocity flow rate of the solution at a steady state. The wall shear stress in the model is calculated using Newton's Law of Viscosity (Equation 5) (Wesenl & Bird, 2006).

$$Newton ’ s Law of Viscosity of flow in \text{x-direction} acting on \text{y-surface}$$

$${τ}_{yx} = μ \frac{{dv}_{x}}{dy} \qquad Equation 5$$

$${τ}_{yx} = wall shear in \text{x-direction} flow acting on \text{y-direction} surface$$

$${v}_{x} = velocity vector in \text{x-direction}$$

$$μ = viscosity of the fluid$$

The results of the wall shear stress simulations are presented in FIG. 15 and FIG. 16. FIG. shows the wall shear stress simulation using Ansys Fluent software with 7E-05 m/s inlet velocity at the surface parallel to the reaction surface. The simulation includes (25) wall shear stress at the inlet port and (26) wall shear stress at the collection port. The simulation indicates that the shear stress at the inlet (25, FIG. 15) and collection port (26, FIG. 15) are significantly higher than those at any other surface of the compartment. This result confirmed the expectation since at inlet port and collection port; the velocity is its highest. However, the shear stress is still minuscule, with a maximum of approximately 4E-05 Pascals at both ports.

FIG. 16 shows the wall shear stress simulation using Ansys Fluent software with 7E-05 m/s inlet velocity, adjusted range, throughout the (19) perfusion compartment. The simulation includes (27) wall shear stress at the reaction surface, (28) wall shear stress at first flow barrier, and (29) wall shear stress at the collection compartment. FIG. 16 displays the result of the same simulation, but the contour's range is adjusted to illustrate the distribution of stress over the compartment. The inlet port and collection port has no contour because their shear stresses are out of the adjusted range. The result shows the shear stress at the reaction surface, the first flow barrier (28, FIG. 16), and the collection compartment (29, FIG. 16) downstream to the second flow barrier.

The wall shear stress result through the perfusion compartment indicated that there is low wall shear stress across the device, as predicted due to low fluid flow velocity. Maximum stress at the inlet is about 6E-05 Pascals, outlet about 3E-05 Pascals. The wall shear stress result through the perfusion compartment indicated that within the device, shear stress is higher close to the inlet port and every uphill and downhill section. Maximum is at the top of the two hills, with a magnitude of 2E-06 Pascals. The wall shear stress result through the perfusion compartment indicated that there is low and uniform shear stress at the reaction surface, ensuring maximum cell retention in the compartment.

### Example 3: Carbon Dioxide Distribution Studies

Carbon dioxide distribution was studied in the main perfusion reactor. It is important to maintain a biologically compatible environment at the reaction surface. Since the device's roof is made using PMMA polymer layer, or other appropriate material listed in Table 1, which maintains gas permeability property, the environment in the device can be assumed to contain 5% Carbon Dioxide if the whole device is placed in a CO₂ incubator. The carbon dioxide distribution can be calculated throughout the liquid film flowing using mass transport constitutive principles. The following assumptions are applied to the simulation: 1) diffusion only (no convective mass transport), 2) constant mass diffusion coefficient, 3) constant fluid properties (Newtonian fluid), 4) steady state (non-transient), 5) no mass flux at any wall of the device (boundary condition at the solid-liquid surface), and 6) 5% Carbon dioxide incubator environment (boundary condition at the liquid-gas surface).

The simulation was based on the diffusion-only mass transport constitutive principle, written in Equation 6, called Fick's second law of diffusion (Wesenl & Bird, 2006).

$$Fick ’ s second law of diffusion$$

$$\frac{\partial {c}_{A}}{\partial t} = {D}_{mix} [ \frac{{\partial }^{2} {c}_{A}}{\partial {x}^{2}} + \frac{{\partial }^{2} {c}_{A}}{\partial {y}^{2}} + \frac{{\partial }^{2} {c}_{A}}{\partial {z}^{2}} ] \qquad Equation 6$$

$${c}_{A} = concentration of carbon dioxide$$

$${D}_{mix} = diffusion coefficient between carbon dioxide and water$$

$$t = time$$

Using these assumptions, the reaction surface carbon dioxide distribution at steady state in a CO₂ incubator can be demonstrated in FIG. 17. FIG. 17 shows carbon dioxide steady-state distribution results simulated with 7E-05 m/s inlet velocity of the surface. The simulation includes (30) carbon dioxide (CO₂) concentration at inlet port, (31) CO₂ concentration at holding compartment, (32) CO₂ concentration at reaction compartment, (33) CO₂ concentration at collection compartment, and (34) CO₂ concentration at collection port, all depicted as an inside view projection. The result indicates that the carbon dioxide concentration at steady state throughout the compartment is uniform and approaching 5%—the level appropriate for bacterial life. Using a gas permeable material for the roof of the compartment allows the incubator's air to diffuse into the device, negating the need to infuse carbon dioxide gas into the medium.

### REFERENCES

L. Valgimigli, A. Baschieri, R. Amorati, Antioxidant activity of nanomaterials, J. Mater. Chem. B. 6 (2018) 2036-2051. doi:10.1039/c8tb00107c.
H. Zazo, C. G. Millán, C. I. Colino, J. M. Lanao, Applications of Metallic Nanoparticles in Antimicrobial Therapy, in: Antimicrob. Nanoarchitectonics From Synth. to Appl., Elsevier Inc., 2017: pp. 411-444. doi:10.1016/B978-0-323-52733-0.00015-X.
W. Mei, Q. Wu, Applications of Metal Nanoparticles in Medicine/Metal Nanoparticles as Anticancer Agents, in: Met. Nanoparticles, Wiley-VCH Verlag GmbH & Co. KGaA, Weinheim, Germany, 2017: pp. 169-190. doi:10.1002/9783527807093.ch7.
A. Fariq, T. Khan, A. Yasmin, Microbial synthesis of nanoparticles and their potential applications in biomedicine, J. Appl. Biomed. 15 (2017) 241-248. doi:10.1016/j.jab.2017.03.004.
M. L. Verma, S. Sharma, K. Dhiman, A. K. Jana, Microbial Production of Nanoparticles: Mechanisms and Applications, in: Nanotechnol. Life Sci., Springer, Cham, 2019: pp. 159-176. doi:10.1007/978-3-030-16383-9_7.
V. Saklani, S. V. K. Jain, Microbial Synthesis of Silver Nanoparticles: A Review, J. Biotechnol. Biomater. s13 (2013) 1-4. doi:10.4172/2155-952x.s13-007.
S. Ravindran, P. Singh, S. Nene, V. Rale, N. Mhetras, A. Vaidya, Microbioreactors and Perfusion Bioreactors for Microbial and Mammalian Cell Culture, in: Biotechnol. Bioeng., IntechOpen, 2019. doi:10.5772/intechopen.83825.
D. Egger, M. Fischer, A. Clementi, V. Ribitsch, J. Hansmann, C. Kasper, Development and characterization of a parallelizable perfusion bioreactor for 3D cell culture, Bioengineering. 4 (2017). doi:10.3390/bioengineering4020051.
J. Schmid, S. Schwarz, R. Meier-Staude, S. Sudhop, H. Clausen-Schaumann, M. Schieker, R. Huber, A perfusion bioreactor system for cell seeding and oxygen-controlled cultivation of three-dimensional cell cultures, Tissue Eng.—Part C Methods. 24 (2018) 585-595. doi:10.1089/ten.tec.2018.0204.
J. D. Robertson, L. Rizzello, M. Avila-Olias, J. Gaitzsch, C. Contini, M. S. MagoÅ, S. A. Renshaw, G. Battaglia, Purification of Nanoparticles by Size and Shape, Sci. Rep. 6 (2016) 1-9. doi:10.1038/srep27494.
S. K. Balasubramanian, L. Yang, L. Y. L. Yung, C. N. Ong, W. Y. Ong, L. E. Yu, Characterization, purification, and stability of gold nanoparticles, Biomaterials. 31 (2010) 9023-9030. doi:10.1016/j.biomaterials.2010.08.012.
Z. Yang, H.-R. Xiong, Culture Conditions and Types of Growth Medium for Mammalian Cells, in: Biomed. Tissue Cult., InTech, 2012. doi:10.5772/52301.
Pump types, World Pumps. 2003 (2003) 40-45. doi:10.1016/s0262-1762(03)90138-6.
Wesenl R. Byron Bird, Transport Phenomena, Revised 2nd Edition, 2006. https://www.wiley.com/en-us/Transport+Phenomena %2C+Revised+2nd+Edition-p-9780470115398 (accessed Jul. 28, 2020).
B. E. Rapp, Microfluidics: Modeling, mechanics and mathematics, Elsevier Inc., 2016. doi:10.1016/c2012-0-02230-2.
Allegheny Ludlum Steel Corporation, Stainless Steel Handbook, 1951. https://www.amazon.com/Stainless-Steel-Handbook-None/dp/B000E1EOCU (accessed Jul. 28, 2020).
Z. Ahmad, Principles of Corrosion Engineering and Corrosion Control, Elsevier Ltd, 2006. doi:10.1016/B978-0-7506-5924-6.X5000-4.
J. G. Michael Naboka, Copper Alloys: Preparation, Properties and Applications, 2011. https://ebookcentral.proquest.com/lib/northeastern-ebooks/detail.action?docID=3021164 (accessed Jul. 28, 2020).
JAB J. A. Brydson, Plastics Materials, 1999. https://ebookcentral.proquest.com/lib/northeastern-ebooks/detail.action?docID=318259 (accessed Jul. 28, 2020).
A. Kozbial, L. Bhandary, B. B. Collier, C. S. Eickhoff, D. F. Hoft, S. K. Murthy, Automated generation of immature dendritic cells in a single-use system, J. Immunol. Methods. 457 (2018) 53-65. doi:10.1016/j.jim.2018.03.010.
G. M. Edelman, Cell adhesion molecules, Science (80-.). 219 (1983) 450-457. doi:10.1126/science.6823544.
Y. Nakai, H. Yoshimizu, Y. Tsujita, Enhancement of Gas Permeability in HPC, CTA and PMMA under Microwave Irradiation, Polym. J. 38 (2006) 376-380. doi:10.1295/polymj.38.376.
P. M. Budd, N. B. McKeown, Highly permeable polymers for gas separation membranes, Polym. Chem. 1 (2010) 63-68. doi:10.1039/b9py00319c.
Ansys Fluent software: (ansys.com/products/fluids/ansys-fluent).
Das, R. K., Pachapur, V. L., Lonappan, L. et al., Biological synthesis of metallic nanoparticles: plants, animals and microbial aspects. Nanotechnol. Environ. Eng. 2, 18 (2017). https://doi.org/10.1007/s41204-017-0029-4

What is claimed is:

1. A perfusion bioreactor for continuous microbial synthesis of metallic nanoparticles, the bioreactor comprising:
(i) an inlet stage comprising a liquid nutrient medium reservoir fluidically coupled to a pump, the pump fluidically coupled to a perfusion compartment;
(ii) the perfusion compartment, comprising
a holding compartment configured for receiving the nutrient medium from the inlet stage, the holding compartment configured for uniform distribution and transfer of the nutrient medium to a reaction compartment;
the reaction compartment configured for formation of said nanoparticles by microbial cells, the reaction compartment comprising a reaction surface suitable for supporting microbial cells used to synthesize the nanoparticles;
a collection compartment configured to collect nutrient medium containing the synthesized nanoparticles from the reaction compartment and transfer the medium and nanoparticles to a collection port;
a first flow barrier disposed between the holding compartment and the reaction compartment, wherein the first flow barrier is operative to uniformly disperse the nutrient medium prior to entry of the medium into the reaction compartment; and
a second flow barrier disposed between the reaction compartment and the collection compartment, wherein the second flow barrier is operative to retain the microbial cells in the reaction compartment and allow the nanoparticles to flow into the collection compartment.
(iii) an outlet stage fluidically coupled to the collection port, the outlet stage operative to collect the nanoparticles and depleted nutrient medium from the collection port;

2. The bioreactor of claim 1, wherein the second flow barrier provides a suspension of nanoparticles to the collection compartment that is substantially free of the microbial cells.

3. The bioreactor of claim 2, wherein the second flow barrier provides a suspension of nanoparticles to the collection compartment that comprises less than 1% (weight/weight) microbial cells.

4. The bioreactor of claim 1, wherein the collection compartment comprises one or more filters.

5. The bioreactor of claim 4, wherein the filter comprises pores having an average diameter of about 0.2 μm.

6. The bioreactor of claim 1, wherein the perfusion bioreactor comprises a gas-permeable roof.

7. The bioreactor of claim 1, wherein the reaction surface is removable.

8. The bioreactor of claim 1, wherein the collection compartment has a triangular shape converging at an apex to the collection port.

9. The bioreactor of claim 1, wherein the first flow barrier provides a flow of the nutrient medium that is slower than the flow of the liquid medium from the inlet stage.

10. A method for synthesizing metallic nanoparticles, the method comprising the steps of:
(a) providing the perfusion bioreactor of any of the preceding claims, a microbial cell culture, a liquid nutrient medium for growth of the microbial cells, and a metal salt;
(b) cultivating microbial cells in the reaction compartment of the perfusion bioreactor in the presence of the metal salt under flow of the liquid nutrient medium through the reaction compartment, whereby said metallic nanoparticles are formed in the liquid nutrient medium and collected in the collection chamber; and
(c) collecting the metallic nanoparticles at the collection port of the collection chamber.

11. The method of claim 10, wherein the microbial cells are bacterial cells.

12. The method of claim 10, wherein step (b) is performed continuously for at least about one week.

13. The method of claim 10, wherein the nanoparticles collected in step (c) are in form of a suspension of nanoparticles that is essentially free of microbial cells from the reaction compartment.

14. The method of claim 13, wherein the suspension of nanoparticles comprises less than 1% (weight/weight) of said microbial cells.

15. The method of claim 10, further comprising filtering the suspension of nanoparticles.

16. The method of claim 15, wherein the filtering comprises a use of a filter comprising pores with an average diameter of about 0.2 μm.

17. A kit for the synthesis of nanoparticles by microbial cells, the kit comprising the perfusion compartment of the perfusion bioreactor of claim 1, and instructions for use thereof.

18. The kit of claim 17, further comprising one or more replacement reaction surfaces of the perfusion compartment.

19. The kit of claim 17, further comprising the inlet stage and/or outlet stage of the perfusion bioreactor.

20. The kit of claim 17, further comprising one or more microbial cell cultures and/or one or more reagents for the production of metallic nanoparticles.
